(19) United States
(12) Patent Application Publication
(10) Pub. No.: US 2023/0022239 A1
(43) Pub. Date: Jan. 26, 2023

(54) LIQUID DRUG PUMPS WITH A FLEXIBLE DRUG RESERVOIR

(71) Applicant: JANSSEN BIOTECH, INC., Horsham, PA (US)

(72) Inventors: Michael CANNAMELA, Atlantic Highland, NJ (US); Peter KRULEVITCH, Pleasanton, CA (US); MingQi ZHAO, Milpitas, CA (US); Brandon MCKENZIE, West Chester, PA (US)

(21) Appl. No.: 17/786,313

(22) PCT Filed: Dec. 2, 2020
(86) PCT No.: PCT/IB2020/061412

§ 371 Date: Jun. 16, 2022

Related U.S. Application Data
(60) Provisional application No. 62/950,337, filed on Dec. 19, 2019.

(51) Int. Cl. A61M 5/142; A61M 5/168; A61M 5/44

(52) U.S. Cl. CPC A61M 5/14216; A61M 5/14248; A61M 5/16804; A61M 5/445; A61M 2005/14208

(57) ABSTRACT

Various exemplary liquid drug pumps with a flexible drug reservoir are provided. In general, a pump includes a flexible reservoir configured to contain a liquid drug therein for delivery to a patient wearing the pump. The reservoir is configured to be filled with the drug from a drug storage container, which can either be external to the pump or disposed within the pump.

### FIELD

The present disclosure relates generally to liquid drug pumps with a flexible drug reservoir.

### BACKGROUND

Pharmaceutical products (including large and small molecule pharmaceuticals, hereinafter “drugs”) are administered to patients in a variety of different ways for the treatment of specific medical indications. A pump is a type of drug administration device that can administer a liquid drug to the patient. Some pumps are wearable by a patient and can include a reservoir, such as a vial or a cartridge, that contains the liquid drug therein for delivery to the patient through a needle inserted into the patient.

The drug can be removed from the reservoir through a conduit and delivered to the patient through the needle. However, if the conduit is not in complete communication with the liquid drug in the reservoir, air can enter the conduit with the drug or instead of the drug. Air is undesirable to deliver to the patient because of, e.g., patient discomfort. If the conduit is not in complete communication with the liquid drug in the reservoir, the patient's desired treatment is interrupted by the pump delivering only air to the patient instead of the drug, by the pump delivering air to the patient with only a partial intended dose of the drug, or by the pump not delivering any air or any drug to the patient due to a detected error of air entering the conduit from the reservoir. Interrupting the patient's treatment may adversely affect the patient's health and may cause patient frustration with the pump and thereby reduce the patient's willingness to use the pump in the future as recommended by the patient's health care provider.

The conduit may not be in complete communication with the liquid drug in the reservoir for a variety of reasons. For example, the conduit may not be in complete communication with the liquid drug in the reservoir due to the patient's orientation when the drug is being pumped out of the reservoir and into the patient via the needle. Liquid in the reservoir naturally settles at a location therein due to gravity, so depending on the patient's orientation, the liquid drug may not settle within the reservoir at a location where the conduit is in complete communication with the liquid drug. Additionally, for pumps that deliver multiple doses of the drug over time, it becomes more likely over time that the patient's orientation will adversely affect the conduit's accessibility of the drug within the reservoir. As the amount of the drug in the reservoir decreases, there is less drug present in the reservoir to be in complete communication with the conduit.

For another example, the conduit may not be in complete communication with the liquid drug in the reservoir due to the pump not being positioned properly on the patient. The pump will typically come with instructions indicating how the pump should be attached to the patient, including a recommended orientation of the pump relative to the patient. The recommended orientation may help maximize the conduit's ability to be in complete communication with the drug in the reservoir for every delivery of the drug to the patient. However, the pump may not be attached to the patient at the recommended orientation due to unintentional user error.

Accordingly, there remains a need for improved liquid drug accessibility.

### SUMMARY

In general, liquid drug pumps with a flexible drug reservoir are provided.

In one aspect, a pump configured to deliver a liquid drug to a patient is provided that in one embodiment includes a flexible reservoir configured to receive the liquid drug from a drug storage container, a rigid chamber configured to receive the drug from the reservoir, an injector assembly configured to receive the drug from the chamber, and control circuitry configured to control pumping of the drug from the reservoir to the chamber and then from the chamber to the injector assembly. The reservoir is configured to expand and collapse. The pump can have any number of variations.

In another aspect, a method of using a pump configured to deliver a liquid drug to a patient is provided and in one embodiment includes causing, using control circuitry of the pump, movement of the liquid drug from a flexible reservoir of the pump, to a rigid chamber of the pump, and from the chamber to an injector assembly for delivery into the patient. The method can have any number of variations.

### BRIEF DESCRIPTION OF DRAWINGS

The present invention is described by way of reference to the accompanying figures which are as follows:

FIG. 1 is a schematic view of an embodiment of a pump configured to deliver a liquid drug to a patient;

FIG. 2 is a schematic view of the pump of FIG. 1 with a drug storage container disposed within the pump;

FIG. 3 is a schematic view of the pump of FIG. 1 with a drug storage container disposed external to the pump;

FIG. 4 is a side cross-sectional view of an embodiment of a reservoir of the pump of FIG. 1,

FIG. 5 is a side cross-sectional view of the reservoir of FIG. 4 coupled to an embodiment of a drug storage container;

FIG. 6 is another side cross-sectional view of the reservoir and the drug storage container of FIG. 5;

FIG. 7 is a side cross-sectional view of the reservoir of FIG. 6 decoupled from the drug storage container and coupled to an injector assembly of the pump of FIG. 1;

FIG. 8 is a side cross-sectional view of another embodiment of a reservoir of the pump of FIG. 1 coupled to an embodiment of a drug storage container;

FIG. 9 is a side cross-sectional view of yet another embodiment of a reservoir of the pump of FIG. 1 coupled to an embodiment of a drug storage container;

FIG. 10 is a perspective cross-sectional view of still another embodiment of a reservoir of the pump of FIG. 1 coupled to an embodiment of a drug storage container;

FIG. 11 is a side cross-sectional view of a metering pump of FIG. 10;

FIG. 12 is a perspective cross-sectional view of another embodiment of a reservoir of the pump of FIG. 1 coupled to an embodiment of a drug storage container;

FIG. 13 is a perspective cross-sectional view of yet another embodiment of a reservoir of the pump of FIG. 1 coupled to an embodiment of a drug storage container;

FIG. 14 is a side view of a portion of FIG. 13;

FIG. 15 is a cross-sectional view of an embodiment of a filling device configured to be used with the pump of FIG. 1;

FIG. 16 is a side cross-sectional view of the filling device of FIG. 15 coupled to an embodiment of a drug storage container;

FIG. 17 is another side cross-sectional view of the filling device and drug storage container of FIG. 16;

FIG. 18 is a side cross-sectional view of the filling device and drug storage container of FIG. 17 coupled to the pump of FIG. 1;

FIG. 19 is a side schematic view of another embodiment of a reservoir of the pump of FIG. 1;

FIG. 20 is a schematic view of another embodiment of a pump configured to deliver a liquid drug to a patient;

FIG. 21 is a schematic view of yet another embodiment of a pump configured to deliver a liquid drug to a patient;

FIG. 22 is an exploded view of a tab and of an electronics module of the pump of FIG. 1; and

FIG. 23 is a perspective view of a printed circuit board of the electronics module of FIG. 22.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary liquid drug pumps with a flexible drug reservoir are provided. In general, a pump includes a flexible reservoir configured to contain a liquid drug therein for delivery to a patient wearing the pump. The reservoir is configured to be filled with the drug from a drug storage container, which can either be external to the pump or disposed within the pump. The reservoir being flexible allows the reservoir to expand in volume as the drug enters the reservoir from the drug storage container to accommodate the drug within the reservoir. The reservoir being flexible also allows the reservoir to collapse in volume as the drug exits the reservoir for delivery to the patient. The reservoir is thus configured to ensure that only drug exits the reservoir for delivery to the patient and, therefore, that the pump's fluid path between the reservoir and the pump's needle or cannula inserted into the patient does not receive therein any air. The patient may thereby be ensured to receive only drug through the needle or cannula, and the patient's drug dose(s) can therefore be fully delivered at a desired schedule without interruption since drug, and not any air, will be provided to the needle or cannula.

The reservoir being flexible allows the reservoir to efficiently occupy space within the pump. The flexible reservoir is configured to expand in proportion to the amount of drug received therein from the drug storage container and to collapse in proportion to the amount of drug exiting the reservoir for delivery to the patient. Thus, the reservoir may occupy as little space as possible within the pump and/or be located in an irregularly shaped area within the pump that a traditionally sized and shape reservoir (such as a vial or cartridge) would not be able to occupy. The flexible reservoir may therefore allow for other components within the pump to be larger and thus more powerful (e.g., a more powerful processor, a more powerful motor, etc.) and/or for mechanisms to be included in the pump that would otherwise not have enough room for inclusion within the pump (e.g., a sensor configured to sense operating condition(s) of the pump, a sensor configured to sense physiological parameter(s) of the patient, a wireless transceiver configured to transmit information regarding the pump to an external receiver and/or to receive information from an external source, etc.).

The reservoir being flexible allows an overall weight of the pump to be reduced, as compared to pumps having rigid reservoirs such as glass or plastic vials or cartridges that weigh more than a flexible reservoir. The flexible reservoir may therefore facilitate a more comfortable experience for the patient wearing the pump.

Some injectable drugs are required to be stored at a cold temperature prior to use, such as by being stored in a refrigerator. Before the drug is injected, the drug should be allowed to warm to room temperature because a cold drug injected into a patient may cause patient discomfort for at least some patients. This warming time can be on the order of minutes, such as at least five minutes, in a range of twenty to thirty minutes, etc. The flexible reservoir may allow for the warm-up time of the drug to be utilized time, in which the reservoir is being filled from the drug storage container, instead of the warm-up time being unutilized waiting time where the user is simply waiting for the drug to warm up without any other activity occurring with respect to the pump.

During reservoir filling, the pump can be in its packaging, e.g., in a tray holding the pump, such that the pump is in a predictable position. The drug from the drug storage container can thus be delivered predictably to the pump without the pump's position preventing or hindering the reservoir from being filled with the drug. The pump can also be in its packaging during priming, in which any air downstream of the reservoir is removed prior to commencing drug delivery to the patient.

In some embodiments, an amount of the drug that is transferred from the drug storage container to the reservoir can be a full amount of the drug within the drug storage container. A known amount of drug may therefore be within the reservoir, ready for delivery to a patient. The pump may therefore be configured for use with any patient, which may facilitate distribution and/or selling of the pump. In other embodiments, an amount of the drug that is transferred from the drug storage container to the reservoir can be an amount calculated based on a weight of a particular patient who will use the pump. This weight-based drug transfer may help ensure that the patient receives no more of the drug than prescribed since the reservoir will not receive therein more drug than prescribed and/or may help ensure that drug is not wasted since only that amount of drug intended for delivery to the patient can be transferred to the reservoir from the drug storage container. In some implementations, any remaining drug in the drug storage container may be used later to re-fill the reservoir or may be used in filling a different reservoir for the same or different patient.

The drug to be delivered using a pump as described herein can be any of a variety of drugs. Examples of drugs that can be delivered using a pump as described herein include antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, antiallergics, antihistamines, anti-inflammatories, corticosteroids, disease modifying anti-rheumatic drugs, erythropoietin, and vaccines.

The flexible drug reservoirs described herein can be used with a variety of drug delivery pumps configured to deliver a drug to a patient. Examples of drug delivery pumps include the pumps described in Intl. Pat. Pub. WO 2018/096534 entitled “Apparatus For Delivering A Therapeutic Substance” published May 31, 2018, in U.S. Pat. Pub. No. 2019/0134295 entitled “Local Disinfection For Prefilled Drug Delivery System” published May 9, 2019, in U.S. Pat. No. 7,976,505 entitled “Disposable Infusion Device Negative Pressure Filling Apparatus And Method” issued Jul. 12, 2011, and in U.S. Pat. No. 7,815,609 entitled “Disposable Infusion Device Positive Pressure Filling Apparatus And Method” issued Oct. 19, 2010, which are hereby incorporated by reference in their entireties. Other examples of drug delivery pumps include the SmartDose® Drug Delivery Platform available from West Pharmaceutical Services, Inc. of Exton, Pa., the OMNIPOD® available from Insulet Corp. of Acton, Mass., the YpsoDose® patch injector available from Ypsomed AG of Burgdorf, Switzerland, the BD Libertas™ wearable injector available from Becton, Dickinson and Co. of Franklin Lakes, N.J., the Sorrel Medical pump available from Sorrel Medical of Netanya, Israel, the SteadyMed PatchPump® available from SteadyMed Ltd. of Rehovot, Israel, the Sensile Medical infusion pump available from Sensile Medical AG of Olten, Switzerland, the SonceBoz wearable injectors available from SonceBoz SA of Sonceboz-Sombeval, Switzerland, enFuse® available from Enable Injections of Cincinnati, Ohio, the on-body injector for Neulasta® available from Amgen, Inc. of Thousand Oaks, Calif., the Pushtronex® System available from Amgen, Inc. of Thousand Oaks, Calif., and the Imperium® pump available from Unilife Corp. of King of Prussia, Pa.

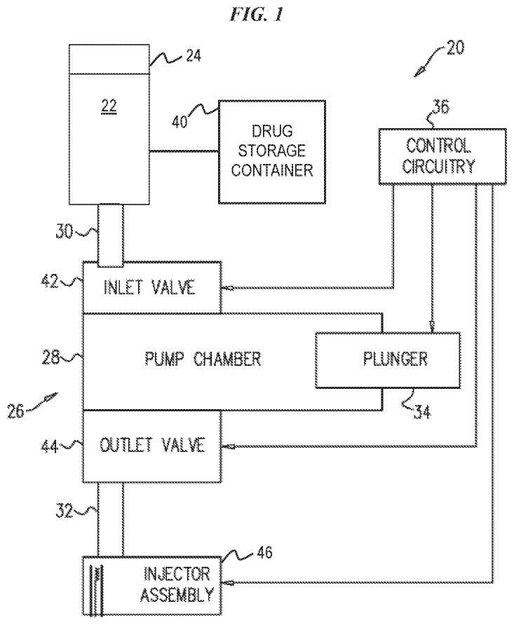

FIG. 1 illustrates an embodiment of a pump 20, e.g., a patch pump, configured to be worn by a patient and to deliver a drug (also referred to herein as a “therapeutic substance”) 22 to the patient. The pump 20 can be configured to be attached to the patient in any of a variety of ways, as will be appreciated by a person skilled in the art, such as by including a backing or label configured to be removed from a body of the pump 20 to expose adhesive attachable to the patient. The pump 20 includes a therapeutic substance reservoir 24 containing the drug 22 therein. The reservoir 24 can be prefilled by a medical vendor or device manufacturer, or the reservoir 24 can be filled by a user (e.g., the patient, the patient's caregiver, a doctor or other health care professional, a pharmacist, etc.) prior to use of the pump 20. As discussed further below, the reservoir 24 is a flexible member configured to receive the drug 22 therein from a drug storage container 40.

The pump 20 also includes an inlet fluid path 30 operatively connected to the reservoir 24 and to an injector assembly 46 of the pump 20 that is configured to deliver the therapeutic substance 22 into a patient. The inlet fluid path 30 includes a tube in which the drug 22 can flow.

The pump 20 also includes an electromechanical pumping assembly 26 operatively connected to the reservoir 24 and configured to cause, e.g., via a motor of the pumping assembly 26, delivery of the therapeutic substance 22 to the patient via the injector assembly 46, e.g., through a needle or cannula of the injector assembly 46 that has been inserted into the patient. The electromechanical pumping assembly 26 is shaped to define a rigid pump chamber 28 that includes a therapeutic substance inlet 30 through which the therapeutic substance 22 is received from the conduit 30, and hence from the reservoir 24, into the pump chamber 28. The rigid pump chamber 28 also includes an outlet fluid path 32 through which the therapeutic substance 22 is delivered from the pump chamber 28 to the patient via the injector assembly 46. Although the pumping assembly 26 is electromechanical in this illustrated embodiment, the pumping assembly of the pump 20 (and for other embodiments of pumps described herein) can instead be mechanical. The mechanical pumping assembly need not include any electronic components or controls. For example, the mechanical pumping assembly can include a balloon diaphragm configured to be activated to cause delivery of a drug through mechanical action.

The pump 20 also includes a plunger 34 slidably disposed within the pump chamber 28 and sealably contacting an inside of the pump chamber 28. The plunger 34 is configured to be in direct contact with the drug 22 in the pumping chamber 28.

The pump 20 also includes control circuitry 36. The electromechanical pumping assembly 26 is configured to be driven to operate in two pumping phases by the control circuitry 36. In a first pumping phase, the control circuitry 36 is configured to drive the plunger 34 (e.g., slidably move the plunger 34 in the pump chamber 28) to draw the drug 22 from the reservoir 24 into the inlet fluid path 30, then through an inlet valve 42 and into the pump chamber 28. The inlet valve 42 is configured to be opened and closed such that when the inlet valve 42 is open there is fluid communication between the reservoir 24 and the pump chamber 28, and when the inlet valve 42 is closed there is no fluid communication between the reservoir 24 and the pump chamber 28. During the first pumping phase, the control circuitry 36 is configured to cause the inlet valve 42 to open, cause an outlet valve 44 to close, and drive the plunger 34 to draw the therapeutic substance 22 from the reservoir 24 into the pump chamber 28, e.g., the control circuitry 36 is configured to set the inlet valve 42 and the outlet valve 44 such that the therapeutic substance 22 can flow only between the reservoir 24 and the pump chamber 28. Thus, as the plunger 34 is drawn back, therapeutic substance 22 is drawn into pump chamber 28. The control circuitry 36 causing the inlet valve 42 to open and the outlet valve 44 to close can be active control or can be passive control in which the valves 42, 44 are mechanical valves that automatically open/close due to the driving of the plunger 34.

In a second pumping phase, the control circuitry 36 is configured to drive the plunger 34 to deliver the drug 22 from the pump chamber 28 through the outlet valve 44 to the outlet fluid path 32 and then to the injector assembly 46 for delivery into the patient. The outlet valve 44 is configured to be opened and closed such that when the outlet valve 44 is open there is fluid communication between the pump chamber 28 and the patient, and when the outlet valve 44 is closed there is no fluid communication between the pump chamber 28 and the patient. During the second pumping phase, the control circuitry 36 is configured to cause the inlet valve 42 to close, cause the outlet valve 44 to open, and drive the plunger 34 to deliver the therapeutic substance 22 from the pump chamber 28 in a plurality of discrete motions of the plunger 34. For example, the control circuitry 36 can be configured to set the inlet valve 42 and the outlet valve 44 such that the therapeutic substance 22 can flow only between the pump chamber 28 and the patient, and the plunger 34 is incrementally pushed back into the pump chamber 28 in a plurality of discrete motions thereby delivering the therapeutic substance 22 to the patient in a plurality of discrete dosages. Similar to that discussed above, the control circuitry 36 causing the inlet valve 42 to close and the outlet valve 44 to open can be active control or can be passive control in which the valves 42, 44 are mechanical valves that automatically open/close due to the driving of the plunger 34.

In some embodiments, the control circuitry 36 is configured to drive the plunger 34 to draw the therapeutic substance 22 into the pump chamber 28 in a single motion of the plunger 34, e.g., plunger 34 is pulled back in a single motion to draw a volume of the therapeutic substance 22 into the pump chamber 28 during the first pumping phase. Alternatively, that control circuitry 36 can be configured to drive the plunger 34 to draw the therapeutic substance 22 into the pump chamber 28 in one or more discrete expansion motions of the plunger 34, e.g., the plunger 34 can be pulled halfway out of the pump chamber 28 in one motion and then the rest of the way out of the pump chamber 28 in a second, separate motion. In this case, a duration of some or all expansion motions of the plunger 34 during the first pumping phase are typically longer than a duration of any one of the plurality of discrete motions of the plunger 34 during the second pumping phase.

In other embodiments, the control circuitry 36 is configured to drive the plunger 34 such that a duration of the first pumping phase and a duration of the second pumping phase are unequal. For example, a duration of the second pumping phase can be in a range of five to fifty times longer than the first pumping phase, e.g., at least ten times, thirty times, fifty times, etc. longer than a duration of the first pumping phase.

The pump 20 can also include a power source (not shown) configured to provide power to the control circuitry 36 and the pumping assembly 26. In an exemplary embodiment, the power source is a single power source configured to provide power to each component of the pump 20 requiring power to operate, which may help reduce cost of the pump 20 and/or conserve space within the pump 20 for other components and/or to help reduce an overall size of the pump 20. The power source can, however, include a plurality of power sources, which may help provide redundancy and/or help reduce cost of the pump 20 since some components, e.g., the control circuitry 36, may be manufactured with an on-board dedicated power supply.

The drug storage container 40 (e.g., a vial or other container such as a cartridge) is either external and releasably connectable to the pump 20 or is disposed within the pump 20. The drug storage container 40 typically has a standardized size with a standardized set amount of the drug 22 contained therein. One-size drug storage containers are generally easier and less costly to manufacture than multiple drug storage containers each having a different amount of the drug 22 contained therein. Examples of drug storage container 40 sizes for cartridges include 5 ml, 10 ml, 20 ml, 30 ml, 40 ml, and 50 ml. Examples of drug storage container 40 sizes for vials include 5R, 10R, 15R, 20R, and 25R.

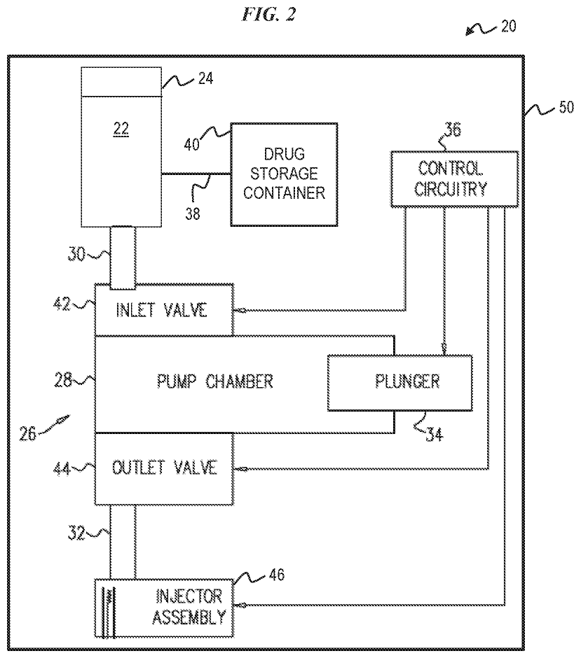

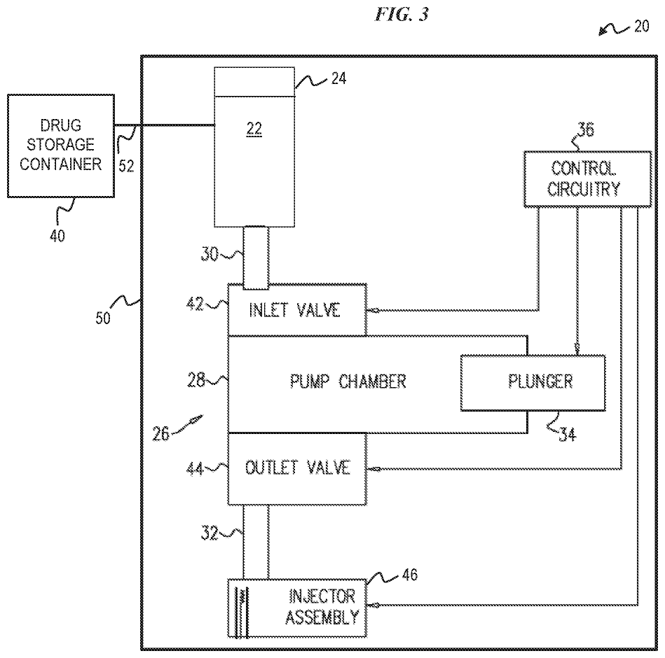

FIG. 2 illustrates an embodiment of the pump 20 in which the drug storage container 40 is disposed in the body 50 of the pump 20. A fluid conduit 38 is operatively connected between the drug storage container 40 and the reservoir 24 to allow the drug 22 to be provided to the reservoir 24 from the drug storage container 40. The pumping assembly 26, e.g., a motor thereof, can be configured, under control of the control circuitry 36, to cause the drug 22 to move from the drug storage container 40 to the reservoir 24. FIG. 3 illustrates an embodiment of the pump 20 in which the drug container 40 is external to the body 50 of the pump 20 and is releasably connectable to the pump 20 via the fluid conduit 38, which is an infusion line 52 in this illustrated embodiment, for delivery of the drug 22 from the drug storage container 40 to the reservoir 24 within the body 50.

The reservoir 24 can have a variety of configurations. In general, the reservoir 24 is a flexible member with elasticity that is configured to expand in volume as the drug 22 enters the reservoir 24 from the drug storage container 40 to accommodate the drug 22 therein and to collapse in volume as the drug 22 exits the reservoir 24 for delivery to the patient via the injector assembly 46. The reservoir 24 is formed of flexible or expandable material to allow for the reservoir's expanding and collapsing. An amount of the reservoir 24 expansion corresponds to a volume of drug 22 moved therein from the drug storage container 40. Examples of the reservoir 24 includes a balloon, a bladder, a coiled tube, and a diaphragm. An example of flexible or expandable materials that can be used for the reservoir includes rubber. An interior surface of reservoir 24 can be coated with a barrier material configured to protect the drug 22 from any damage that could potentially be caused to the drug 22 by contact with the material of the reservoir 24. An example of a barrier material includes FluroTec® film available from West Pharmaceutical Services, Inc. of Exton, Pa.

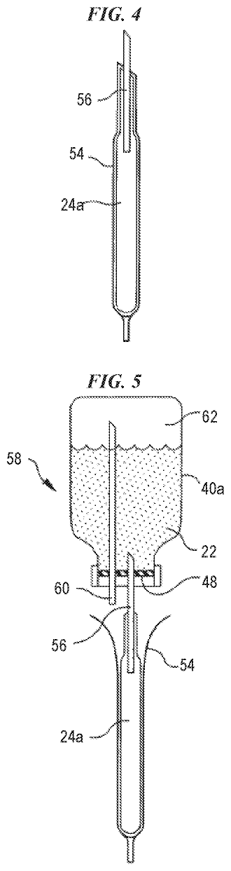

FIG. 4 illustrates an embodiment of the reservoir 24 as a bladder 24a. The bladder 24a in an initial position, in which the bladder 24a contains no drug therein, is confined within an enclosure 54. FIG. 4 shows the bladder 24a in the initial position. The bladder 24a includes a needle 56, also within the enclosure 54, that communicates with an interior of the bladder 24a. As shown in FIG. 5, the needle 56 forms part of a filling system 58 for filling the bladder 24a. The filling system 58 also includes the drug storage container 40 (which is a vial 40a in this illustrated embodiment but as mentioned above can have another form, such as a cartridge) and a vent tube 60. The vial 40a includes a septum 48 through which the needle 56 and the vent tube 60 extend. The vent tube 60 extends to above a fill line of the drug 22 into a head space 62.

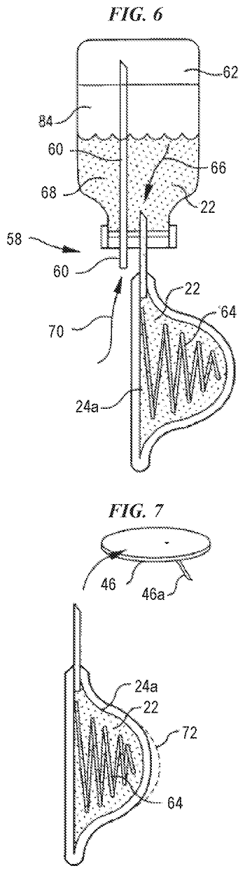

FIG. 6 shows the bladder 24a being filled. The needle 56 of the bladder 24a has been inserted through the septum 48 of the vial 40a. The filling system 58 further includes a spring 64, as shown in FIG. 6 that is compressed when the bladder 24a is confined within the enclosure 54, as shown in FIG. 4 (the spring 64 is obscured in FIG. 4). However, when the enclosure 54 is unsealed, as shown in FIG. 5, permitting the needle 56 of the bladder 24a to penetrate the septum 48 and the enclosure 54 is removed from the bladder 24a, the bladder 24a will expand, causing the drug 22 to be drawn into the bladder 24a from the vial 40a. More specifically, when the bladder 24a expands under the influence of the internal expander formed by the spring 64, a negative pressure is created within the bladder 24a causing the drug 22 to flow into the needle 56 of the bladder 24a in the direction indicated by reference numeral 66 and into an internal area of the bladder 24a. The spring 64 can be tuned for the desired amount of drug 22 volume to fill the bladder 24a. The volume of liquid medicament displaced 68 is replaced by air drawn in to the vial 40a through the vent tube 60 in the direction indicated by reference numeral 70. When the bladder 24a is fully expanded, the bladder 24a will be filled with the drug 22.

FIG. 7 schematically illustrates the bladder 24a in use after being filled with the drug 22. The bladder 24a is coupled to the pumping assembly 26 for drawing the drug 22 from the bladder 24a against the expansion force of the spring 64. Instead of the spring 64 being within the bladder 24a as shown in FIGS. 6 and 7, the spring 64 can be located within the pump 20 outside of the bladder 24a. Alternatively, the spring 64 can be omitted such that the bladder 24a expands and collapses under its own force.

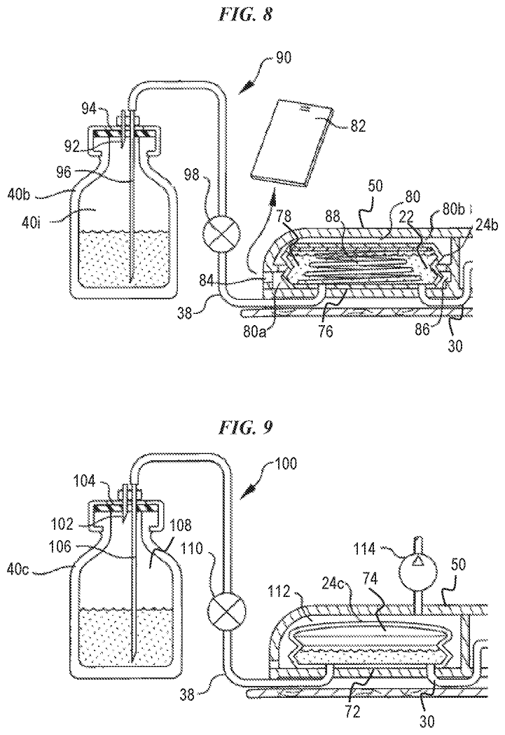

FIG. 8 illustrates an embodiment of the reservoir 24 as a diaphragm 24b. The diaphragm 24b is mounted for stability to an internal surface 76 of the pump 20 within the housing 50 of the pump 20. The fluid conduit 38 and the inlet fluid path 30 are in operative communication with an internal area 78 of the diaphragm 24b that is configured to contain the drug 22 therein. The diaphragm 24b is disposed and confined within a chamber 80 in the housing 50 of the pump 20. The chamber 80 includes a lower chamber 80a and an upper chamber 80b. A volume of the chamber 80 is initially determined by a substantially rigid removable panel 82 originally bridging the chamber 80 and confined by a slot 84 and a recess 86 to define the lower chamber 80a. A person skilled in the art will appreciate that an element, such as the panel 82, may not be slightly less than fully rigid but nevertheless be considered to be substantially rigid due to any number of factors such as manufacturing tolerances and sensitivity of measurement equipment. When the panel 82 is removed through the slot 84, the diaphragm 24b expands into the upper chamber 80a under the influence of a spring 88, similar to that described above regarding the bladder 24a and the spring 64. The diaphragm 24b will now occupy substantially all of the chamber 80. A person skilled in the art will appreciate that an element, such as the diaphragm 24b, may not occupy precisely all of a space, such as the chamber 80, but nevertheless be considered to occupy substantially all of the space due to any number of factors such as manufacturing tolerances and sensitivity of measurement equipment.

The diaphragm 24b of FIG. 8 is being filled by a filling system 90. The spring 88 forms part of the filling system 90 for filling the diaphragm 24b. The filling system 90 also includes the drug storage container 40 (which is a vial 40b in this illustrated embodiment but as mentioned above can have another form, such as a cartridge) and a vent tube 92. The vial 40b includes a septum 94 through which a filling needle 96 and the vent tube 92 extend. The vent tube 92 vents an interior 40i of the vial 40b to atmospheric pressure. The diaphragm 24b is coupled to the filling needle 96 by the conduit 38 and a pressure controlled valve 98.

When the diaphragm 24b is to be filled with the drug 22 from the vial 40b, the pump 20 is coupled to the filling needle 96 by inserting the conduit 38 into the diaphragm 24b. Then, the panel 82 is removed and the diaphragm 24b expands under the influence of the spring 88, which creates a negative pressure within the internal area 78 of the diaphragm 24b that is translated to the conduit 38 causing the valve 98 to open. The drug 22 will now flow into the filling needle 96, through valve 98, and through conduit 38 into the diaphragm 24b. The volume of drug 22 displaced within the vial 40b is replaced by air drawn in through the vent tube 92. When the diaphragm 24b is fully expanded, a set volume of the drug 22 has been transferred to the diaphragm 24b, and the filling procedure is completed. The pump 20 filled with the drug 22 is now ready for use. To that end, the pump 20 can be detached from the filling assembly 90 and adhered to a patient's skin as discussed herein. In use, the diaphragm 24b is configured to provide the drug 22 to the inlet fluid path 30 under the influence of the pumping assembly 26 of the pump 20, which, when actuated, draws the drug 22 from the diaphragm 24b against the expansion force of the spring 88 and provides the drug 22 to the inlet fluid path 30.

FIG. 9 illustrates another embodiment of the reservoir 24 as a diaphragm 24c. The diaphragm 24c is mounted for stability to an internal surface 72 of the pump 20 within the housing 50 of the pump 20. The fluid conduit 38 and the inlet fluid path 30 are in operative communication with an internal area 74 of the diaphragm 24c that is configured to contain the drug 22 therein.

The diaphragm 24c of FIG. 9 is being filled by a filling system 100. The filling system 100 includes the drug storage container 40 (which is a vial 40c in this illustrated embodiment but as mentioned above can have another form, such as a cartridge) and a vent tube 102. The vial 40c includes a septum 104 through which a filling needle 106 and the vent tube 102 extend. The vent tube 102, similar to that discussed above, vents an interior area 108 of the vial 40c to atmospheric pressure. The filling system 100 also includes the conduit 38 and a pressure controlled valve 110, which couples to the diaphragm 24c to the filing needle 106.

A chamber 112 within the housing 50 of the pump 20 a has a fixed volume in this illustrated embodiment. The pump's chamber 112 is coupled to a vacuum pump 114 of the filling system 100. Whereas the diaphragm 24b of FIG. 8 is configured to be expanded under the influence of the spring 88, the diaphragm 24c of FIG. 9 is configured to be expanded under the influence of a vacuum applied to the chamber 112, and hence to the diaphragm 24c disposed therein, by the external vacuum pump 114.

When the diaphragm 24c is to be filled with the drug 22 from the vial 40c, the pump 20 is coupled to the filling tube 106 by inserting the conduit 38 into the diaphragm 24c. Then, the vacuum pump 114 is activated, which causes the diaphragm 24c to expand under the influence of the vacuum pulled in the chamber 112. Thus, the diaphragm 24c is expanded by means external to the diaphragm 24c. The expansion of the diaphragm 24c creates a negative pressure within the diaphragm 24c that is translated to the conduit 38, causing the valve 110 to open. The drug 22 will now flow into the filling needle 106, through conduit 38 and through valve 110 and into the diaphragm 24c. Similar to that discussed above regarding FIG. 8, a volume of the drug 22 displaced from the vial 40c is replaced by air drawn in through the vent tube 102. When the diaphragm 24c is fully expanded, the filling procedure is completed. The pump 20 is now ready for use to deliver the drug 22 to a patient as discussed herein.

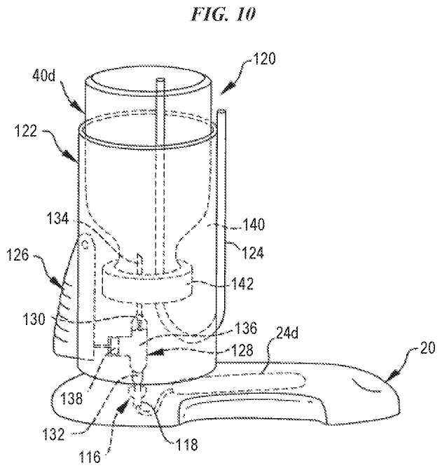

FIG. 10 illustrates an embodiment of the reservoir 24 as a balloon 24d. The pump 20 includes a fill port 116 configured to facilitate filling of the balloon 24d with drug from the drug storage container 40, which in this illustrated embodiment is a vial 40d but as mentioned above can have another form, such as a cartridge. The fill port 116 can include a septum (not shown) that configured to be pierced by a needle 118 carried by a filling device 120 during the filling of the balloon 24d. The filling device 120 includes a substantially cylindrical housing 122, a vent tube 124, an actuator 126, and a metering pump 128. A person skilled in the art will appreciate that an element, such as the housing 122, may not be precisely cylindrical but nevertheless be considered to be substantially cylindrical due to any number of factors such as manufacturing tolerances and sensitivity of measurement equipment. The metering pump 128, which is also shown in FIG. 11, includes a first one-way valve 130, a second one-way valve 132, a needle 134, a piston chamber 136, and a piston 138. The second one-way valve 132 can have any of a variety of valve forms, such as a drip-less, excellent flow, low volume valve, such as a swabable luer valve.

The filling device's housing 122 has a cavity 140 dimensioned to receive the vial 40d therein. When the vial 40d is received within the filling device's housing 122, an end cap 142 of the vial 40d is pierced first by the vent tube 124 and then by the needle 134. A length of the vent tube 124 is selected so that when the vial 40d is fully received within the housing 122, the end of the vent tube 124 extends above the drug 22. The vent tube 124 is thus configured to permit the drug 22 to flow freely from the vial 40d, through the first one-way valve 130, and into the chamber 136 of the metering pump 128. When the actuator 126 is depressed, the piston 138 is caused to exert a direct positive pressure on the drug 22 within the piston chamber 136 to displace a set volume or known quantity of the drug 22 from the piston chamber 136. The set volume or known quantity of displaced liquid medicament flows through the second one-way valve 132, and through the needle 118 into the balloon 24d. Hence, the number of times that the actuator 126 is depressed determines the amount of the drug 22 transferred from the vial 40d to the balloon 24d. This controllability of drug 22 transfer amount from the vial 40d to the balloon 24d allows the balloon 24d to be filled with a precise and desired amount of the drug 22. Filling the reservoir 24 with a precise and desired amount of the drug 22 may be useful for, e.g., weight-based dosing in which a particular amount of drug 22 is provided from the drug storage container 40 to the reservoir 24.

In use, the needle 118 is attached to the second one-way valve 132 of the metering pump 128. Next, the vial 40d is placed in the cavity 140 of the filling device's housing 122 and the filling device 120 is removably attached to the pump 20. Next, the actuator 126 is depressed the number of times required to transfer the desired quantity of the drug 22 from the vial 40d to the fill port 116 and thus the balloon 24d. When the desired quantity of the drug 22 has been transferred to the pump 20 from the vial 40d, the filling device 120 is removed from the pump 20, the needle 118 is removed from the second one-way valve 132. The filling device 120 can then be placed in sterile storage with the vial 40d remaining in the housing 30. Such storage of the vial 40d supports multiple use of the filling device 120.

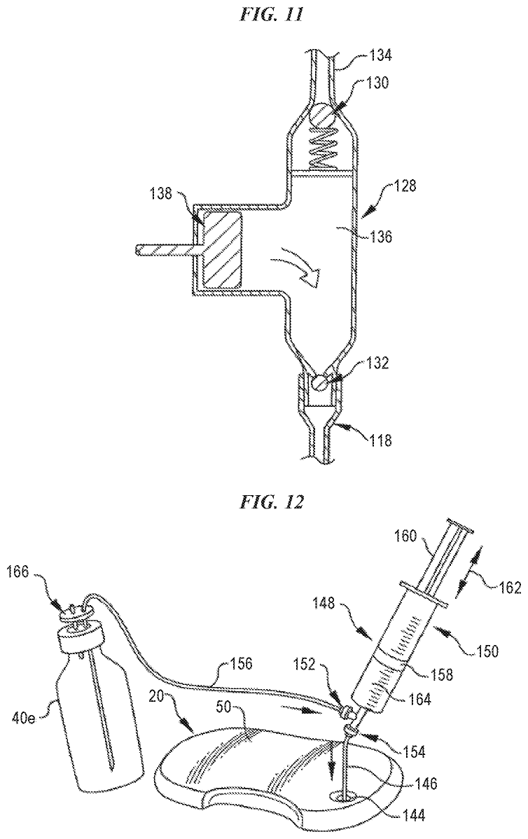

FIG. 12 illustrates another embodiment of the reservoir 24 as a balloon (obscured by the pump 20 in FIG. 12). A fill port 144 of the pump 20 is configured to receive a fill tube 146 carried by a filling device 148 during the filling of device reservoir. The filling device 148 includes a syringe 150, a first one-way valve 152, a second one-way valve 154, and a transfer tube 156. The transfer tube 156 couples to an interior of the drug storage container 40 (which is a vial 40e in this illustrated embodiment but as mentioned above can have another form, such as a cartridge) to the first one-way valve 152, which permits the drug 22 to be drawn from the vial 40d as a piston 158 of the syringe 150 is withdrawn through movement of an actuator 160 of the syringe 150 in a first direction. Possible actuator 160 movement is represented by arrows 162. As the piston 158 is withdrawn, a chamber 164 is formed of a known volume that is filled with the drug 22. The drug 22 can flow freely due to a vacuum release or vent tube 166. When the chamber 164 is expanded to hold a desired or set volume of the drug 22, the actuator 160 is moved in a second, opposite direction to cause the piston 158 to exert a direct positive pressure on the drug 22. The drug 22 thus flows from the syringe chamber 164 through the fill tube 146 and into the fill port 144 of the pump 20. When the volume of the chamber 164 has been diminished completely, the known or set volume of drug 22 has been transferred to the pump 20, e.g., the balloon thereof.

FIG. 13 illustrates another embodiment of the reservoir 24 as a balloon (obscured by the pump 20 in FIG. 13). In this illustrated embodiment, a filling device 168 includes a substantially cylindrical housing 170, a vent tube 172, an actuator 174, and a metering pump 176. The housing 170 of the filling device 168 has a cavity 178 configured to receive therein the drug storage container 40, which is a vial 40f in this illustrated embodiment but as mentioned above can have another form, such as a cartridge. When the vial 40f is received within the cavity 178 of the housing 170, an end cap 180 of the vial 40f is pierced first by the vent tube 172 and then by a needle 182. A length of the vent tube 172 is selected so that when the vial 40f is fully received within the housing 170, the end of the vent tube 172 extends above the drug 22. The vent tube 172 thus vents the vial 40f to atmospheric pressure to permit the drug 22 to flow freely from the vial 40f.

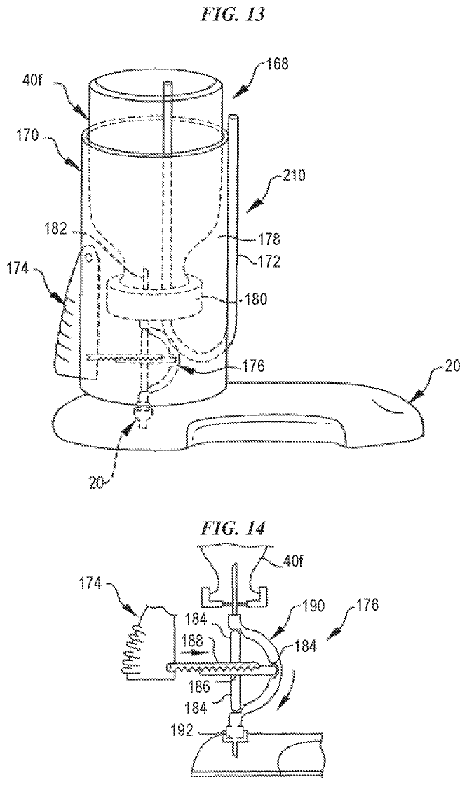

The metering pump 176 is a peristaltic pump in this illustrated embodiment and is also shown in FIG. 14. The metering pump 176 includes a plurality of radially extending rotating fingers 184. The fingers 184 are configured to rotate about a toothed hub 186. The teeth of the toothed hub 186 are configured to be driven by teeth of a toothed drive member 188 connected to the actuator 174. A transfer tube 190 conducts the drug 22 from the vial 40f to a fill port 192 of the pump 20. The fingers 184 are configured to rotate when the actuator 174 is depressed. The ends of the rotating fingers 184 engage the transfer tube 190 to push the drug 22 to the fill port 192. Each depression of the actuator 174 meters a set volume of the drug 22 to the fill port 192, similar to that discussed above regarding the actuator 126. In this embodiment, the drug 22, although receiving direct positive pressure from the metering pump 176, is never actually touched by pump mechanism.

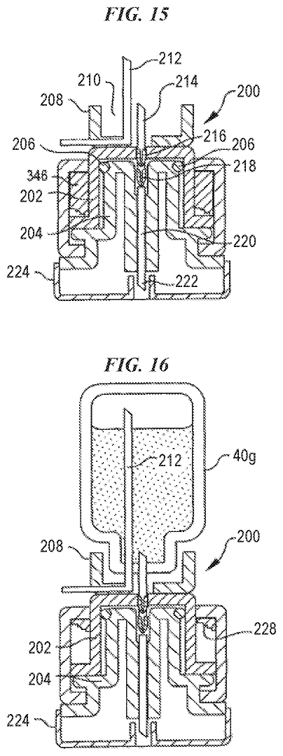

FIGS. 15-18 illustrate another embodiment of the reservoir 24 as a balloon 24g. In this illustrated embodiment, a filling device 200 includes a plunger 202 that reciprocates on a frame 204. Seal rings 206 provide a seal between the plunger 202 and the frame 204. On top (in the perspective illustrated in FIGS. 15-18) of the plunger 202 is a ring 208 that defines a cavity 210 configured to receive the drug storage container 40, which in this illustrated embodiment is a vial 40g but as mentioned above can have another form, such as a cartridge. The filling device 200 also includes a vent tube 212 and a transfer tube 214. A first one-way valve 216 is configured to permit the drug 22 to be transferred to an intermediate chamber 226 when the plunger 202 is withdrawn. A second one-way valve 218 permits the drug 33 to flow from the intermediate chamber 226 into a fill tube 220. The fill tube 220 has an end 222 that is received by the pump's fill port. The filling device 200 further includes a protective cover 224 that protects the fill tube 220 during filling device 200 storage.

FIG. 16 shows the filling device 200 after the ring 208 has received the vial 40g. The vent tube 212 is venting the vial 40g to atmospheric pressure above the drug 22.

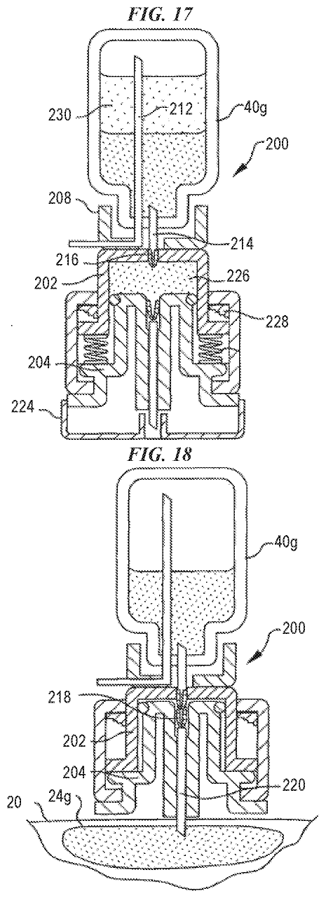

In FIG. 17, the plunger 202 has been withdrawn, thereby causing the drug 22 to flow from the vial 40g, through the transfer tube 214 and the first one-way valve 216, into the intermediate chamber 226 formed by the withdrawal of the plunger 202. An extent in which the plunger 202 is withdrawn and a volume of the drug 22 to be transferred is set by a spacer 228. The spacer 228 includes two rings joined by a stepped incline. Depending upon which relative directions the rings of the spacer 228 are rotated with respect to each other, the spacer 228 is widened or narrowed to control the travel of the plunger 202, and hence the volume of the drug 22 transferred to the intermediate chamber 226. Here, the volume of the drug 22 so transferred is seen at 230.

In FIG. 18, the protective cover 224 has been removed and the filling device 200 has been coupled to the pump 20 for filling the balloon 24g. The plunger 202 has been brought to its initial position thus completely reducing the intermediate chamber 226, which has caused, through direct positive, the drug 22 to have flowed from the intermediate chamber 226, through the second one-way valve 218 and the fill tube 220 into the balloon 24g of the pump 20. The filling of the balloon 24g is now complete, and the protective cover 224 can be once again placed on the filling device 200 for storage.

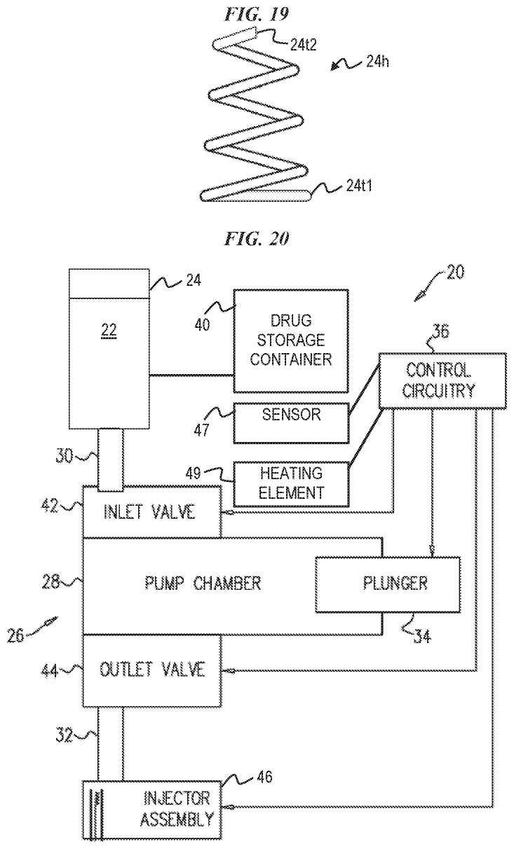

FIG. 19 illustrates an embodiment of the reservoir 24 as a coiled tube 24h. The coiled tube 24h can have any number of coils. Due to the coiled tube's placement within the pump 20, e.g., due to space constraints within the pump 20, one or more of the coiled tube's coils may not have a coil shape within the pump 20 but instead may be more linearly positioned. A first terminal end 24t1 of the coiled tube 24h is connected to the drug storage container 40 and is configured to receive the drug 22 therethrough from the drug storage container 40. A second terminal end 24t2 of the coiled tube 24h is connected to the inlet fluid path 30 and is configured to pass the drug 22 therethrough to the inlet fluid path 30. Similar to that discussed above, the pumping assembly 26, e.g., a motor thereof, is configured, under control of the control circuitry 36, to cause the drug 22 to move from the drug storage container 40 to the coiled tube 24h and from the coiled tube 24h to the pump chamber 28.

Referring again to FIG. 1, regardless of a type of the reservoir 24 and how the reservoir 24 is filled, the control circuitry 36 can be configured to prevent the needle or cannula of the injector assembly 46 to be inserted into the patient until after the filling of the reservoir 24 has been completed. For example, the pump 20 can include a sensor configured to monitor a fill volume of the reservoir 24. The sensor can be in operative communication with the control circuitry 36 which, based on the fill volume, can be configured to determine whether or not the reservoir 24 is full. For another example, the control circuitry 36 can be in operative communication with a sensor of the drug storage container 40 configured to monitor a fill volume of the drug storage container 40. The control circuitry 36 can, based on the fill volume, be configured to determine whether or not the reservoir 24 is full. For another example, the control circuitry 36 can include a clock or other timer configured to determine if a predetermined threshold amount of time has passed since the drug 22 began filling the reservoir 24 from the drug storage container 40. The predetermined amount of time can be based on a size of the drug storage container 40, and therefore on an amount of the drug 22 in the drug storage container 40, as such sizes are typically standardized. Alternatively, the predetermined amount of time can be based on an amount to be transferred from the drug storage container 40 to the reservoir 24 in a weight-based dosing scheme.

FIG. 20 illustrates an alternate embodiment of the pump 20 of FIG. 1 that is the same as the pump 20 of FIG. 1 except that the pump 20 of FIG. 20 also includes a sensor 47 and a heating element 49. The sensor 47 and a heating element 49 are each in operative communication with the control circuitry 36. The heating element 49 is configured to heat the drug 22 in the reservoir 24. As discussed above, some drugs should be allowed to warm to room temperature before being injected, and in such a case, the heating element 49 may speed up the warming of the drug 22. The patient may therefore have less waiting time before using the pump 20, which reduces frustration. The heating element 49 can have a variety of configurations, such as a heating coil, a heating cable, a positive temperature coefficient (PTC) heater, or a resistive element configured to warm as current passes therethrough.

The heating element 49 can be at a variety of locations. For example, the heating element 49 can be wrapped around the reservoir 24, e.g., wrapped one or more times around an exterior perimeter of the reservoir 24. For another example, the heating element 49 can be located against a bottom exterior surface of the reservoir 24, where the inlet fluid path 30 is considered to extend from a top of the reservoir 24. For yet another example, the heating element 49 can be located against an exterior surface of the reservoir 24 that is configured to face the ground when the pump 20 is attached to a patient at the pump's recommended orientation and the patient is at an expected upright posture, whether standing or sitting. In this way, the drug 22 settled in the reservoir 24 due to gravity will be settled near the heating element 49 and therefore may be more effectively warmed by the heating element 49 should the heating element 49 be turned on with the pump 20 attached to the patient. For another example, the heating element 49 can be located against an exterior surface of the reservoir 24 that is configured to face the ground when the pump 20 is in its packaging and the packaging is resting on a table, a shelf, or other substantially flat surface. In this way, the drug 22 settled in the reservoir 24 due to gravity will be settled near the heating element 49 and therefore may be more effectively warmed by the heating element 49 should the heating element 49 be turned on before the pump 20 is removed from its packaging. A person skilled in the art will appreciate that the surface may not be precisely flat but nevertheless be considered to be substantially flat due to any number of factors such as manufacturing tolerances and sensitivity of measurement equipment. For yet another example, the heating element 49 can be located at least partially within the reservoir 24. The heating element 49 may therefore be in direct contact with the drug 22 in the reservoir 24, which may speed warming of the drug 22 by the heating element 49 as compared to implementations in which the heating element 49 is not in direct contact with the drug 22, e.g., by the heating element 49 being located entirely outside of the reservoir 24.

The control circuitry 36 is configured to turn the heating element 49 on (providing heat) and off (not providing heat). The control circuitry 36 can be configured to turn on the heating element 49 for a predetermined amount of time, e.g., an amount stored in a memory of the control circuitry 36. The predetermined amount of time can be based on one or more factors, such as on a type of the drug 22 and/or on a volume of drug 22 in the reservoir 24. The heating element 49 being on for the predetermined amount of time limits heating of the drug 22 by the heating element 49, which may help prevent overheating the drug 22.

The sensor 47 can include a temperature sensor configured to sense a temperature of the drug 22 in the reservoir 24 and to communicate sensed temperature data to the control circuitry 36. The control circuitry 36 can be configured to turn on the heating element 49 when the sensed temperature is below a predetermined minimum threshold temperature and to turn off the heating element 49 when the sensed temperature is above a predetermined maximum threshold temperature. In an exemplary embodiment the temperature sensor is a single sensor, which may help reduce cost of the pump 20, help conserve space within the pump 20 for other components, and/or help reduce an overall size of the pump 20. The temperature sensor can, however, include a plurality of sensors, which may help provide redundancy and allow for temperature measurements to be confirmed with one another for accuracy.

In addition to or instead of the sensor 47 including a temperature sensor, the sensor 47 can include an orientation sensor configured to monitor an orientation of the pump 20 relative to gravity, e.g., the ground. Examples of an orientation configured to monitor orientation include an accelerometer, an inertial measurement unit (IMU), and a MARG (magnetic, angular rate, and gravity) sensor. In an exemplary embodiment the orientation sensor is a single sensor, which may help reduce cost of the pump 20, help conserve space within the pump 20 for other components, and/or help reduce an overall size of the pump 20. The orientation sensor can, however, include a plurality of orientation sensors, which may help provide redundancy and allow for orientation measurements to be confirmed with one another for accuracy. In embodiments in which the sensor 47 includes a temperature sensor and an orientation sensor, the temperature and orientation sensors can be separate sensors or can be integrated together into a single sensor, e.g., as a single sensor chip.

The control circuitry 36 can be configured to not turn on the heating element 49 unless the pump 20 is at an orientation, as indicated by the pump's current orientation as measured by the orientation sensor, within a predefined range of predetermined acceptable orientations. The range of predetermined acceptable orientations is defined by an area of accessibility for the conduit 38 being in complete communication with the drug 22 in the reservoir 24. The range of predetermined acceptable orientations is stored in a memory of the control circuitry 36 for operative access by a processor of the control circuitry. In embodiments in which the sensor 47 includes a temperature sensor and an orientation sensor, the control circuitry 36 can thus be configured to only turn on the heating element 49 when the pump 20 is within the predefined range of predetermined acceptable orientations and when the sensed temperature is below the predetermined minimum threshold temperature.

The control circuitry 36 can be configured to only turn the heating element 49 on before any drug delivery begins, which may help ensure that the heating element 49 is only providing heat during the drug's initial warm-up time after being delivered to the reservoir 24 from the drug storage container 40. Alternatively, the control circuitry 36 can be configured to turn the heating element 49 on at any time after the drug 22 has started to be delivered to the reservoir 24 from the drug storage container 40, which may help ensure that the drug 22 is always at a comfortable temperature when delivered to the patient from the reservoir 24.

As mentioned above, the liquid drug that can be delivered by any of the pumps of FIGS. 1-20 can be any of a variety of drugs. In some embodiments, the liquid drug can include contaminant particles (also referred to herein as “particulates”) therein. The particles can clog the various pathways in which the drug flows and hinder, if not prevent entirely, flow of the drug and thus delivery of the drug to the patient. The pump can therefore include at least one filter along the drug's flow path that is configured to filter the particulates while allowing the liquid of the drug to flow therethrough. The at least one filter can therefore help prevent the particulates from flowing further down the drug's flow path and causing a clog.

Each of the one or more filters can have a variety of sizes, such as 1 micron, 3 micron, 5 micron, etc.

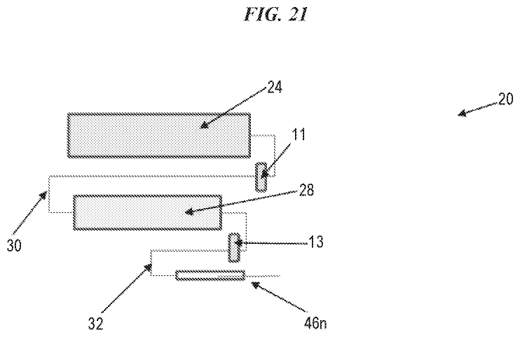

The at least one filter can be located in a variety of locations. FIG. 21 illustrates an alternate embodiment of the pump 20 of FIG. 1 that is the same as the pump 20 of FIG. 1 except that the pump 20 of FIG. 21 also includes a first filter 11 and a second filter 13. The first filter 11 is located along the inlet flow path 30 between the reservoir 24 and the pump chamber 28 (either before or after the inlet valve 42), and the second filter 13 is located along the outlet flow path 32 between the pump chamber 28 and the needle of the injector assembly 46 (either before or after the outlet valve 44). Thus, to the extent that any particles are transferred from the drug storage to container 40 to the reservoir 24, the first filter 11 is configured to reduce an amount of the particles that flow from the reservoir 24 to the pump chamber 28. Similarly, to the extent that any particles are transferred from the reservoir 24 to the pump chamber 28, the second filter 13 is configured to reduce an amount of the particles that flow from the pump chamber 28 to the needle 46n.

In another embodiment, the pump 20 can be similar to the pump 20 of FIG. 21 but omit the second filter 13. In yet another embodiment, the pump 20 can be similar to the pump 20 of FIG. 21 but omit the first filter 11. In yet another embodiment, the pump 20 can be similar to the pump 20 of FIG. 21 but include a third filter along the flow path between the drug storage container 40 and the reservoir 24. In still another embodiment, the pump 20 can be similar to the pump 20 of FIG. 21 but include a filter along the flow path between the drug storage container 40 and the reservoir 24 and include only one of the first and second filters 11, 13.

For the embodiments of FIGS. 1-21, an amount of the drug 22 that is transferred from the drug storage container 40 to the reservoir 24 can be an amount calculated based on a weight of a particular patient who will use the pump 20. This weight-based drug transfer may help ensure that the patient receives no more of the drug 22 than prescribed since the reservoir 24 will not receive therein more drug 22 than prescribed and/or may help ensure that drug 22 is not wasted since only that amount of drug 22 intended for delivery to the patient can be transferred to the reservoir 24 from the drug storage container 40. In at least some instances, any remaining drug 22 in the drug storage container 40 may be used later to re-fill the reservoir 24 or may be used in filling a different reservoir of a different pump for the same or different patient.

The weight-based dose for a patient can be stored in a memory of the control circuitry 36. For safety reasons, a medical professional (e.g., doctor, nurse, etc.) or a pharmacist but not a patient can be allowed to set the weight-based dose. Once the weight-based dose is stored in the memory, the dose setting can be locked so as to be unable to be changed, which may help ensure patient safety.

The control circuitry 36 can be configured to ensure that only the amount of the drug 22 that corresponds to a total amount of drug 22 to be delivered to the patient from the pump 20 is transferred from the drug storage container 40 to the reservoir 24. For example, in the embodiment of FIG. 4, the needle 56 can include a valve therein configured to be selectively opened and closed by the control circuitry 36. The control circuitry 36 can be configured to close the valve when an amount of the drug 22 moved from the drug storage container 40 to the reservoir 24 reaches the total amount of drug 22 to be delivered to the patient from the pump 20. The amount of the drug 22 in the reservoir 24 can be known by the control circuitry 36 by, e.g., a sensor in operative communication with the control circuitry 36 being configured to sense a fill level of the reservoir 24. For another example, in the embodiments of FIGS. 8 and 9, the conduit 38 can similarly include a valve therein configured to be selectively opened and closed by the control circuitry 36.

The pump 20 can include a user interface configured to indicate the weight-based dose for a patient stored in a memory of the control circuitry 36. The user interface can have a variety of configurations, and the pump 20 can include more than one type of user interface. For example, the user interface can include a plurality of lights, e.g., a light emitting diode (LED) or other type of light, configured to illuminate to provide an indication of the set weight-based dose. Each of the lights can correspond to a particular possible dose. An illuminated one of the lights indicates which one of the possible doses has been set. The light can remain illuminated throughout use of the pump 20 to allow the dose to always be easily identified. For another example, the user interface can include a display configured to show thereon an indication of the set weight-based dose, such as by using text. The display can include a display screen having any of a variety of configurations, such as a cathode ray tube (CRT), a liquid crystal display (LCD), a touchscreen, etc.

Instead of using a weight-based dosing scheme in which an amount of the drug 22 that is transferred from the drug storage container 40 to the reservoir 24 is an amount calculated based on a weight of a particular patient who will use the pump 20, the pump 20 can be configured with a lockout dosing scheme. Using a lockout dosing scheme, the pump 20 is configured to prevent delivery of the drug 22 from the reservoir 24 after the amount calculated based on the weight of the particular patient who will use the pump 20 has been delivered to the patient. The patient may therefore receive no more of the drug 22 than prescribed since the patient will not receive more drug 22 from the pump 20 than prescribed. The lockout dosing scheme may simplify transfer of the drug 22 from the drug storage container 40 to the reservoir 24 since, regardless of the particular weight or identity of the patient who receives the pump 20 for use, substantially all of the drug 22 will be transferred from the drug storage container 40 to the reservoir 24. A person skilled in the art will appreciate that all of the drug 22 may not be transferred to the reservoir 24 but nevertheless be considered to have been substantially all transferred due to any number of factors such as manufacturing tolerances and sensitivity of measurement equipment. The patient's weight is still be taken into account under the lockout dosing scheme by the pump 20 being programmable to only deliver the weight-based total amount of the drug 22 to the patient and thereafter locking out delivery of the drug 22. The drug storage container 40 can therefore be the same size with the same amount of drug 22 therein for use with every patient and every pump 20, with dosing for each particular pump 20 being customizable for the particular patient who will use that pump 20, which may facilitate manufacturing and/or selling of the drug storage container 40.

The lockout dosing scheme can be implemented similar to that discussed above regarding the weight-based dosing scheme. The weight-based dose for a patient can be stored in a memory of the control circuitry 36. For safety reasons, a medical professional (e.g., doctor, nurse, etc.) or a pharmacist but not a patient can be allowed to set the weight-based dose. Once the weight-based dose is stored in the memory, the dose setting can be locked so as to be unable to be changed, which may help ensure patient safety. Similar to that discussed above regarding the weight-based dosing scheme, the pump 20 can include a user interface configured to indicate the weight-based dose for a patient stored in a memory of the control circuitry 36.

The control circuitry 36 can be configured to lockout the pump 20 for drug 22 delivery after the amount of the drug 22 that corresponds to the stored total amount of drug 22 to be delivered to the patient from the pump 20 has been delivered to the patient, e.g., has been pumped out of the reservoir 24. For example, in the embodiment of FIG. 4, the needle 56 can include a valve therein configured to be selectively opened and closed by the control circuitry 36. The control circuitry 36 can be configured to maintain the valve in the closed position, e.g., to not re-open the valve, after the stored total amount of drug 22 has been delivered to the patient from the pump 20. The amount of the drug 22 in the reservoir 24 can be known by the control circuitry 36 by, e.g., a sensor in operative communication with the control circuitry 36 being configured to sense a fill level of the reservoir 24. For another example, in the embodiments of FIGS. 8 and 9, the conduit 38 can similarly include a valve therein configured to be maintained in the closed position by the control circuitry 36 after the stored total amount of drug 22 has been delivered to the patient from the pump 20. For yet another example, the control circuitry 36 can be configured to disable the pump's power supply after the stored total amount of drug 22 has been delivered to the patient from the pump 20 so as to prevent re-activation of the pumping assembly 26. The pump 20 can be configured to disable the power supply by, e.g., causing a switch to open that, when closed, operatively connects the power supply to a motor of the pumping assembly 26. For still another example, the control circuitry 36 can be configured to cause mechanical blockage or crimping of a neck of the reservoir 24 (at an end of the reservoir 24 closest to the inlet fluid path) to prevent fluid exit from the reservoir 24. The pump 20 can thus include a movable lock controllable by the control circuitry 36 that moves to block or crimp the reservoir's neck.

For the embodiments of FIGS. 1-21, whether or not a weight-based dosing scheme, a lockout dosing scheme, or neither a weight-based dosing scheme or a lockout dosing scheme are used, the pump 20 can be in its packaging during filling of the reservoir 24 from the drug storage container 40. As mentioned above, the drug 22 from the drug storage container 40 can thus be delivered predictably to the pump 20 without the pump's position preventing or hindering the reservoir 24 from being filled with the drug 22.

The packaging for the pump 20 can include an outer container, e.g., a cardboard box, a plastic box, etc., and a holder, e.g., a tray, a clamshell case, etc., within the box (or other outer container) in which the pump 20 is seated. In some embodiments, the pump 20 can be configured to automatically start the transfer process of moving the drug 22 from the drug storage container 40 to the reservoir 24 in response to the pump 20, in its holder, being removed from the outer container. In such embodiments, the pump 20 and the outer container can be operatively coupled to a tab configured to facilitate the automatic starting of the drug transfer process. In response to the pump 20 being manually removed from the outer container, the tab is configured to be de-coupled from the pump 20 to trigger the pump 20, e.g., the control circuitry 36 thereof, to start the drug transfer process from the drug storage container 40 to the reservoir 24. The de-coupling of the tab from the pump 20 is configured to “wake up” the pump 20 by allowing the pump's power source to begin providing power to the control circuitry 36 and the pumping assembly 26 to allow the control circuitry 36 to cause the pumping assembly 26 to begin the movement of the drug 22 from the drug storage container 40 to the reservoir 24. The tab is thus configured to prevent powered components of the pump 20, e.g., the control circuitry 36, the pumping assembly 26, the pump's user interface (if present), etc., from receiving power until the pump 20 is removed from the outer container. The tab may thus help ensure that the power source is not depleted of power before the pump 20 is used by a patient and/or may allow the power source to be relatively small and/or inexpensive since power only need be provided for after the pump 20 has been removed from the outer container and not during storage of the pump 20 before use.

The power source is configured to not provide power to the pump's powered components when the tab is coupled to the pump 20 and is configured to provide power when the tab is not coupled to the pump 20. The tab is configured to move from a first position, in which the tab is coupled to the pump 20 (corresponding to the power source not providing power and the pump 20 being in the outer container), to a second position, in which the tab is not coupled to the pump 20 (corresponding to the power source 330 providing power and the pump 20 being outside the outer container). With the tab in the first position, the tab acts as an insulator that creates an open circuit that prevents the power source from providing power. The tab is made from an insulating material to allow the tab to act as an insulator. With the tab in the second position, the tab creates a closed circuit that allows the power source to provide power. The control circuitry 36 is configured to start the drug transfer process from the drug storage container 40 to the reservoir 24 in response to being powered on.

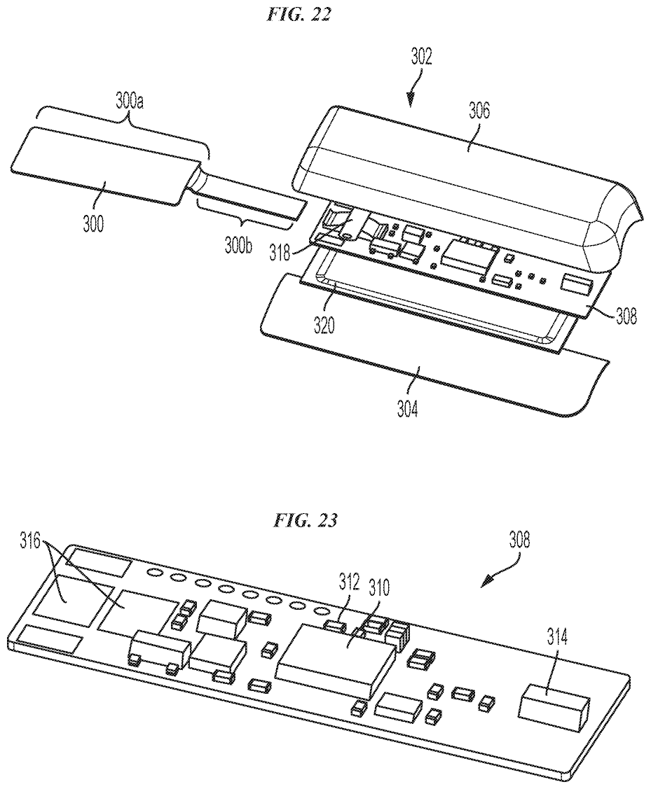

FIG. 22 illustrates an embodiment of a tab 300 and an electronics module 302. The electronics module 302 is part of the pump 20. The electronics module 302 includes a housing defined by the bottom housing portion 304 and a top housing portion 306 that are fixed together. A printed circuit board (PCB) 308, also shown in FIG. 23, is disposed in the housing and supports the pump's control circuitry 36. The PCB 308 in this illustrated embodiment is rigid, although the PCB 308 may instead be flexible. The PCB 308 in this illustrated embodiment includes a processor 310, a memory 312, a communication interface 314 in the form of a chip antenna (although other types of communication interfaces are possible), switch contact pads 316, and a switch 318. Also, a power source 320 of the pump 20 is disposed within the housing.

The tab 300 can have a variety of sizes, shapes, and configurations. In this illustrated embodiment, the tab 300 has a first portion 300a located outside of the electronics module 302 and attached to the outer container, such as by being adhered thereto with adhesive or other attachment mechanism. The tab 300 has a second portion 300b extending from the first portion 300a and extending into the electronics module 302, e.g., into the housing of the electronics module 302. The second portion 300b of the tab 300 is positioned so as to prevent the switch 318 from engaging the switch contact pads 316. In this way, when the tab 300 is removed from the electronics module 302 and is no longer located within the housing of the electronics module 302, the tab 300 no longer prevents the switch 318 from engaging the switch contact pads 316, e.g., closing the open circuit that exists when the tab 300 is in the first position.

The tab 300 being attached to the outer container facilitates movement of the tab 300 from the first position to the second position. When the pump 20 is manually removed by a user from the outer container, the tab 300 attached to the outer container is also removed from the pump 20, thereby de-coupling the tab 300 from the electronics module 302 that is attached to the pump 20. The tab 300 is thus configured to move from the first position to the second position in response to removal of the pump 20 from the outer container. A user therefore need not take any special action to activate the power source 320, e.g., cause the power source 320 to start providing power, since removing the pump 20 from the outer container is a normal part of using the pump 20.

As in this illustrated embodiment, the tab 300 can be configured as a tamper resistant feature. The tab 300 being absent but the pump 20 being in the outer container may be evidence of tampering, e.g., evidence that the pump 20 was removed at some prior time and then replaced back into the outer container. Similarly, the tab 300 being attached to the outer container without the tab's second portion 300b located in the housing of the electronics module 302 may be indicative of tampering, evidence that the pump 20 was removed from the outer container at some prior time and then replaced back into the outer container.

In embodiments in which the holder of the pump 20 inside the outer container is a clamshell case, the removal of the pump in the clamshell case from the outer container can trigger a time-release feature. The control circuitry 36 can be configured to prevent the clamshell case from being opened until a predetermined amount of time has elapsed since the pump 20 and the clamshell case have been removed from the outer container, e.g., since the control circuitry 36 began receiving power from the power source. As mentioned above, the control circuitry 36 can include a clock or other timer configured to determine if a predetermined amount of time has passed. Preventing the clamshell case from being opened for the predetermined amount of time may help ensure that enough time has passed for the intended amount of drug 22 to be transferred from the drug storage container 40 to the reservoir 24 before the pump 20 is attached to the patient. The control circuitry 36 can be configured to prevent the clamshell case from opening in a variety of ways. For example, the control circuitry 36 can be operatively connected to a switch that in a closed, locked position prevents clamshell case opening and in an open, unlocked position allows clamshell case opening.

Instead of starting the drug transfer process in response to removal of the pump from the outer container, in embodiments in which the holder includes a clamshell case, the pump 20 can be configured to automatically start the transfer process of moving the drug 22 from the drug storage container 40 to the reservoir 24 in response to opening of the clamshell case. In such embodiments, instead of the first portion 300a of the tab 300 being attached to the outer container, the first portion 300a of the tab 300 is attached to the clamshell case and is configured to be removed from the electronics module 302 in response to the opening of the clamshell case.

In other embodiments, the pump 20 can be configured to automatically start the transfer process of moving the drug 22 from the drug storage container 40 to the reservoir 24 in response to manual pulling of the tab 300 out of the housing. In such embodiments, the first portion 300a of the tab 300 is not attached to the outer container or to the holder but is instead freely accessible to a user after the pump 20 has been removed from the outer container and, in some embodiments, also from the holder. The tab 300 being manually movable provides more freedom to the user by allowing the user to decide when the pump 20 should begin preparing for drug delivery by moving the drug 22 from the drug storage container 40 to the reservoir 24.

As discussed herein, one or more aspects or features of the subject matter described herein, for example components of the control circuitry and the user interface, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

The present disclosure has been described above by way of example only within the context of the overall disclosure provided herein. It will be appreciated that modifications within the spirit and scope of the claims may be made without departing from the overall scope of the present disclosure.

1. A pump configured to deliver a liquid drug to a patient, comprising:
a flexible reservoir configured to receive the liquid drug from a drug storage container, the reservoir being configured to expand and collapse;
a rigid chamber configured to receive the drug from the reservoir;
an injector assembly configured to receive the drug from the chamber; and
control circuitry configured to control pumping of the drug from the reservoir to the chamber and then from the chamber to the injector assembly.

2. The pump of claim 1, wherein the flexible reservoir is one of a balloon, a bladder, a coiled tube, and a diaphragm.

3. The pump of claim 1, wherein the flexible reservoir is disposed in the pump.

4. The pump of claim 1, further comprising the drug storage container.

5. The pump of claim 4, wherein the drug storage container is non-removably disposed in the pump.

6. The pump of claim 4, wherein the drug storage container is configured to be moved from outside of the pump to inside of and non-removably disposed in the pump.

7. The pump of claim 4, wherein the drug storage container is external to the pump and is removably attachable to the pump.

8. The pump of claim 1, further comprising a heating element configured to heat the drug in the reservoir.

9. The pump of claim 8, wherein the control circuitry is configured to selectively turn the heating element on and off.

10. The pump of claim 1, wherein the pump is seated in a holder configured to hold the pump in a predictable position during the receipt of the drug from the drug storage container.

11. The pump of claim 10, wherein the holder is contained within an outer storage container.

12. The pump of claim 1, further comprising at least one filter along a flow path of the drug, the at least one filter being configured to filter particles from the drug.

13. The pump of claim 1, wherein the pump is configured to be worn by a patient.

14. The pump of claim 1, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

16. A method of using the pump of claim 1, comprising:
causing, using the control circuitry, movement of the liquid drug from the reservoir and into the patient.

17. The method of claim 15, further comprising, prior to causing the movement of the drug, causing, using the control circuitry, heating of the drug in the reservoir using a heating element.

18. The method of claim 17, wherein the control circuitry is configured to cause the heating of the drug only after the pump has been removed from an outer storage container.

19. The method of claim 18, wherein the control circuitry is configured to cause the heating of the drug only after the pump has been removed from the outer storage container and after a clamshell case holding the pump therein has been opened.

20. The method of claim 15, further comprising, prior to causing the movement of the drug, preventing, using the control circuitry, a clamshell case holding the pump therein from being opened until a predetermined amount of time has passed since the clamshell case has been removed from an outer storage container.

21. The method of claim 15, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.
